(19) United States
(12) Patent Application Publication
(10) Pub. No.: US 2012/0266326 A1
(43) Pub. Date: Oct. 18, 2012

(54) FREEZE TOLERANT EUCALYPTUS

(75) Inventors: Chunsheng Zhang, Summerville, SC (US); Brian Kwan, Whakatane (NZ); Shujun Chang, Summerville, SC (US); Peter Raymond, Cottageville, SC (US)

(73) Assignee: ArborGen Inc.

(21) Appl. No.: 13/085,146

(22) Filed: Apr. 12, 2011

(51) Int. Cl. A01H 5/00; C12N 15/82; D21H 11/00; C11B 1/00

(52) U.S. Cl. 800/289; 800/298; 554/8; 162/91

(57) ABSTRACT

The present disclosure relates to freeze-tolerant transgenic Eucalyptus, including methodology for making and products produced there from. Following exposure to cold temperature, and compared to a control plant, the freeze-tolerant Eucalyptus displays a freeze-tolerant phenotype characterized by at least one of increased height, less leaf damage, less dieback of main stem, less crown defoliation, and/or improved tree stem form.

### SEQUENCE LISTING

The instant application contains a Sequence Listing which has been submitted in ASCII format via EFS-Web and is hereby incorporated by reference in its entirety. Said ASCII copy, created on May 2, 2011, is named 04446309.txt and is 27,213 bytes in size.

### FIELD OF TECHNOLOGY

The present disclosure relates to Eucalyptus with a freeze-tolerant phenotype, including methodology for producing same and products derived there from.

### INTRODUCTION

The pulp and paper industry is a major economic sector in the southeastern United States, with annual shipments of paper products valued at almost $60 billion. Hardwood frees in the Southeast are a critical feedstock component for this industry. In addition, the growing need for renewable energy and fuel in the United States is creating a new market for wood as a feedstock for the production of bioenergy and biofuels. A reliable. high quality and cost-effective hardwood supply is necessary to sustain the pulp and paper industry in the United States, both to meet domestic demands and retain a competitive position in global markets. Hardwood supplies in the United States are projected to experience increasing demands, both from the pulp and paper sector as well as emerging new bioenergy applications. Despite this, hardwoods are not extensively planted and managed in dedicated stands due in part to the cost of plantation establishment, and their relatively slow growth and corresponding long rotation time to harvest.

Eucalyptus species are among the fastest growing woody plants in the world and represent about 8% of all planted forests (˜18 million hectares) grown in 90 countries (FAO, 2007). While there are over 700 Eucalyptus species identified, only a limited number are grown commercially. Eucalyptus is a preferred fiber source for the global pulp and paper industry both for its fiber qualities and productivity. It has been the focus of extensive breeding and tree improvement programs aimed at capturing desirable wood properties such as basic density, cellulose content, fiber length and improved growth (Raymond, 2002).

As a native of warm weather climates, the most productive Eucalyptus species favor tropical to sub-tropical conditions, and the preferred fast-growing pulp species show very limited tolerance to freezing temperatures. Attempts have been made to grow a wide variety of Eucalyptus species in several parts of the southeastern US but in many cases these species have been unable to withstand the dramatic and sudden drops in temperature that are typical of the region. Efforts to improve the freezing tolerance of fast growing species through controlled crossing with inherently freeze-tolerant (but slower growing) temperate Eucalyptus species have not been successful. Currently, large scale plantings of Eucalyptus in the southeastern US are limited to regions of central and southern Florida.

### SUMMARY

In one aspect, provided is a transgenic Eucalyptus displaying a freeze-tolerant phenotype. In one embodiment, the transgenic Eucalyptus is a hybrid, such as a E. grandis×E. urophylla. In another embodiment, the freeze-tolerant phenotype is any one or combination of increased height, less leaf damage, less dieback of main stem, less crown defoliation, and/or improved tree stem form (single dominant leader stem), relative to a control plant. In another embodiment, wood, wood fiber, pulp, paper, or oil may be produced from transgenic Eucalyptus.

In another aspect, there is a a method of conferring freezing tolerance in Eucalyptus, comprising (a) introducing into a Eucalyptus cell a construct comprising an isolated polynucleotide sequence operably linked to a promoter, wherein said isolated polynucleotide encodes a CBF polypeptide and wherein the promoter drives the expression of the CBF polypeptide Eucalyptus upon exposure to cold for a period of time; (b) culturing the transformed Eucalyptus cell under conditions promoting growth of a transgenic Eucalyptus tree, thereby producing a transgenic tree; (c) subjecting the transgenic Eucalyptus tree to cold acclimation; (d) exposing the transgenic tree to a freezing temperature for a period of time; and (e) allowing the transgenic tree to recover under conditions that promote growth of the transgenic tree.

In one embodiment, the promoter is Arabidopsis thaliana rd29A promoter. In another embodiment, the freezing temperature is from about 0° C. to about −30° C. In another embodiment, the period of time is from about 2 hours to about 72 hours. In yet another embodiment, the isolated polynucleotide sequence is selected from the group consisting of SEQ ID NO: 1, SEQ ID NO: 3, SEQ ID NO: 5 and SEQ ID NO: 7, or a fragment or variant thereof having at least 70% sequence identity to SEQ ID NO: 1, SEQ ID NO: 3, SEQ ID NO: 5, or SEQ ID NO: 7.

### BRIEF DESCRIPTION OF THE DRAWINGS

FIG. 1: Schematic diagram of vector pABCTE01.

FIG. 2: CBF2 transcript analysis of Eucalyptus hybrid variety '427. RNA samples were reverse transcribed and then PCR amplified using CBF2-specific primers. Lane 1—molecular weight marker; lane 2—RT-PCR sample from line 427; lane 3—No-RT sample from line 427; lane 4—RT-PCR sample from non-transgenic EH1 control; lane 5—No-RT sample from nontransgenic control; lane 6—RT-PCR control with no RNA template.

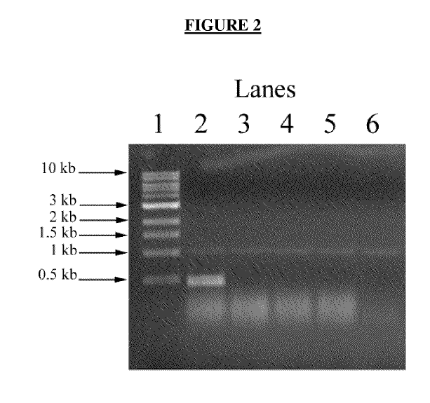

FIG. 3: Photograph of control (non-transgenic) Eucalyptus EH1. Photo panel (A) shows the tree in January. Photo panel (B) shows the tree in March. All leaves on the EH1 control tree are dead and desiccated in January and have been lost by March.

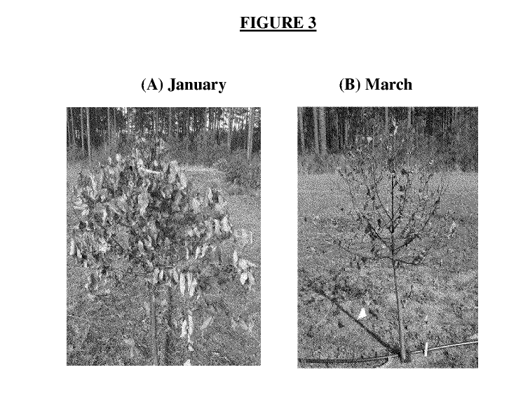

FIG. 4: Photograph of transgenic Eucalyptus line '427. Photo panel (A) shows the tree in January. Photo panel (B) shows the tree in March.

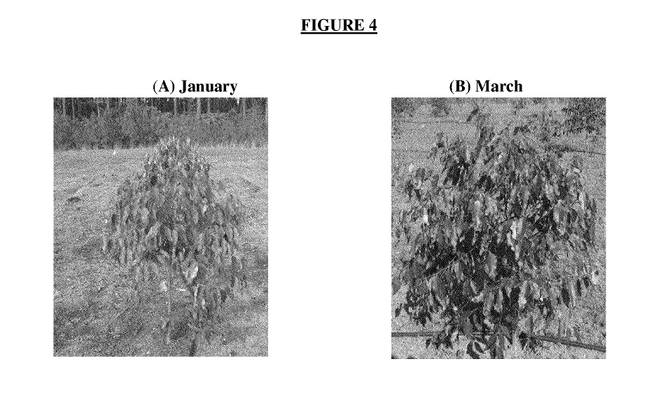

### DETAILED DESCRIPTION

Scientific advancements in understanding the cold acclimation process allowed the discovery of transcription factor genes common to the plant cold-response pathway (Jaglo-Ottsen et al., 1998; Stockinger et al.; 1997, Gilmour et al., 1998; Liu et al., 1998; Kasuga et al., 1999). The discovery of cold tolerant genes combined with the development of efficient Argobacterium-mediated gene transfer methods for Eucalyptus species has allowed the development of genetically engineered freeze-tolerant Eucalyptus (FTE) lines, such as the instant ‘FTE 427.’

Based on the understanding of scientific advances in the freeze tolerance pathway, the present inventors hypothesized that introducing the C-Repeat Binding Factor (CBF2)CBF gene into a fast growing but freeze susceptible commercial genotype of Eucalyptus could enable these trees to withstand freezing events typically experienced in areas found in USDA cold-hardiness zones 8 and 9 in the southeastern United States.

As described below, illustrative freeze-tolerant Eucalyptus was developed by introducing the C-Repeat Binding Factor (CBF2) gene from Arabidopsis into a fast growing but freeze susceptible commercial hybrid genotype of E. grandis×E. urophylla. Of course, and within the spirit of the present disclosure, other freeze-tolerant Eucalyptus plants could be developed; the present application is not limited to the illustrative E. grandis×E. urophylla.

All technical terms in this description are commonly used in biochemistry, molecular biology and agriculture, respectively, and can be understood by those skilled in the field of this invention. Those technical terms can be found in: MOLECULAR CLONING: A LABORATORY MANUAL, 3rd ed., vol. 1-3, ed. Sambrook and Russel, Cold Spring Harbor Laboratory Press, Cold Spring Harbor, N.Y., 2001; CURRENT PROTOCOLS IN MOLECULAR BIOLOGY, ed. Ausubel et al., Greene Publishing Associates and Wiley-Interscience, New York, 1988 (with periodic updates); SHORT PROTOCOLS IN MOLECULAR BIOLOGY: A COMPENDIUM OF METHODS FROM CURRENT PROTOCOLS IN MOLECULAR BIOLOGY, 5^{th} ed., vol. 1-2, ed. Ausubel et al., John Wiley & Sons, Inc., 2002; GENOME ANALYSIS: A LABORATORY MANUAL, vol. 1-2, ed. Green et al., Cold Spring Harbor Laboratory Press, Cold Spring Harbor, N.Y., 1997.

Methods involving plant biology techniques are described herein and are described in detail in methodology treatises such as METHODS IN PLANT MOLECULAR BIOLOGY: A LABORATORY COURSE MANUAL, ed. Maliga et al., Cold Spring Harbor Laboratory Press, Cold Spring Harbor, N.Y., 1995. Various techniques using PCR are described, e.g., in Innis et al., PCR PROTOCOLS: A GUIDE TO METHODS AND APPLICATIONS, Academic Press, San Diego, 1990 and in Dieffenbach and Dveksler, PCR PRIMER: A LABORATORY MANUAL, 2^{nd} ed., Cold Spring Harbor Laboratory Press, Cold Spring Harbor, N.Y., 2003. PCR-primer pairs can be derived from known sequences by known techniques such as using computer programs intended for that purpose (e.g., Primer, Version 0.5, 1991, Whitehead Institute for Biomedical Research, Cambridge, Mass.). Methods for chemical synthesis of nucleic acids are discussed, for example, in Beaucage and Caruthers, Tetra. Letts. 22: 1859-1862 (1981) and Matteucci and Caruthers, J. Am. Chem. Soc. 103: 3185 (1981).

Cold Acclimation

Plants from tropical regions have little to no capacity to withstand freezing temperatures, while plants from temperate regions can survive freezing temperatures ranging from −5 to −30° C. (˜23 to −22° F.), depending on the species. The capacity of plant freeze tolerance is not constitutive, but is induced by exposure to low and non-freezing temperatures (generally below ˜12° C. or ˜54° F.), a phenomenon known as “cold acclimation”.

A significant advance in understanding cold acclimation has been the discovery of the C-repeat/dehydration-responsive element binding factor (CBF/DREB) cold-response pathway in Arabidopsis (Jaglo-Ottsen et al., 1998; Stockinger et al., 1997; Gilmour et al., 1998; Liu et al., 1998; Kasuga et al., 1999). RNA analysis shows that CBF transcripts can be detected in Arabidopsis 1 hour after exposure to cold (4° C., ˜39° F.)) and peaking after 2 hour exposure (Liu et al., 1998) but disappearing after 6 hours, suggesting that their expression is transiently induced by low temperatures. In the majority of studies CBF gene expression appears to be specific to cold induction and does not respond to other stress signals such as ABA, drought or salt stress (Liu et al.1998; Medina et al., 1999).

CBF Sequences

The CBF genes are transcription factors that belong to the AP2/EREBP family of DNA-binding proteins (Riechmann and Meyerowitz, 1998) and like other transcription factors act as control switches for the coordinated expression of other genes in defined metabolic pathways. CBE protein recognizes and binds to a cold- and drought-responsive DNA regulatory sequence designated as the C-repeat (CRT) dehydration-responsive element (DRE) (Baker et al., 1994; Yamaguchi-Shinozaki and Shinozaki, 1994) which is found in the promoter regions of many cold-inducible genes (Maruyama et al., 2004). Both cDNA and microarray experiments identified a variety of genes that function downstream of and are regulated by CBF (Maruyama et al., 2004; Fowler and Thomashow, 2002; Seki et al., 2002; Vogel at al, 2005). All of these genes are involved in functions that mitigate environmental stresses. The CBF genes appear to have redundant functional activities since analysis of transcript levels of other genes revealed no difference between plants over-expressing CBP1, CBF2, or CBF3 (Gilmour et al., 2004; Cook et al, 2004; Fowler and Thomashow, 2002). The changes in gene expression patterns in response to cold could be largely mimicked by ectopic expression of CBF genes at warm temperatures, demonstrating a prominent role of CBF genes in the regulation of cold-response pathways (Cook et al., 2004). CBF genes themselves are regulated by other transcription factors (Zhu et al., 2007; Chinnusamy et al., 2003; Agarwal et al., 2006; Zarka et al., 2003; Zhu et al., 2007). A comparison of CBF-like gene expression in plants that are able to acclimate and those that are unable to acclimate in response to low temperatures concluded that the components of the CBF-cold response pathway are highly conserved in flowering plants and are not limited to those that cold acclimate (Jaglo et al., 2001).

Recent studies have reported that Eucalyptus CBE homologues in species with known cold tolerance are responsive to cold. Transcription of two CBE homologues in Eucalyptus gunnii was detected 15 minutes after exposure to low temperature (4° C.) and reached maximum levels 2-5 hours after exposure (El Kayal et al., 2006). Similarly RT-PCR analysis of a CBE homologue from E. globulus revealed that expression was transiently induced in seedlings 15 minutes after exposure to cold (Gamboa et al., 2007). Two CBE homologues have been isolated from E. dunnii (ArborGen, unpublished results). Transcripts of the E. dunnii CBF homologues were detected in young plants 30 minutes after exposure to low temperature (4° C.), and the cold induction continued up to 4 hours. Over-expression of either of these genes conferred cold tolerance in transgenic Arabidopsis (ArborGen, unpublished results). These results strongly suggest that a functional cold tolerance pathway regulated by CBF exists in some Eucalyptus species. These results also suggest that the susceptibility of tropical Eucalyptus to freezing temperatures may be due to either a lack of and/or an inappropriate expression of specific transcription factors or their target stress tolerance effector genes. While it is expected that the genes for the cold tolerance pathway are present broadly in the Eucalyptus genus, since this pathway does not confer any selective advantage in tropical regions, its functionality has been lost in those Eucalyptus species that are native to tropical regions.

Over-expression of CBF genes have been shown to confer cold, drought and salt tolerance in Arabidopsis (Liu et al., 1998; Kasuga et al., 1999). Over-expression of the Arabidopsis CBF genes in Brassica napus and tobacco induced the expression of orthologs of Arabidopsis CBF-targeted genes and increased the freezing and drought tolerance of transgenic plants (Jaglo et al., 2001; Kasuga et al., 2004). Similar results have been observed from over-expression of Arabidopsis CBE1 in other species including Populus (Benedict et al., 2006). Likewise, CBE homologues have been isolated from a wide variety of species including pepper (Yi et al., 2004), rice (Dubouzet et al., 2003; Ito et al., 2006), maize (Qin et al., 2004) and wheat (Jaglo et al., 2001;Vagujfalvi et al., 2005; Kobayashi et al., 2005), with several of these demonstrating enhanced cold tolerance when transferred into other species. In contrast, there are also some examples where introducing different CBE genes did not lead to increased cold tolerance, particularly in tomato and potato (Hsieh et al., 2002; Zhang et al., 2004, Benham et al., 2007; Pino et al., 2007).

Promoters

The potential for reduced growth by over expression of CBF genes in FTE lines has been significantly mitigated by the use of a cold-inducible promoter that limits the expression of the CBF gene under conditions where expression would be undesirable.

While any cold-inducible promoter may be used, the present application uses the rd29A promoter, which is a cold-inducible promoter from Arabidopsis thaliana. Yamaguchi-Shinozaki and Shinozaki, 1993.

Vector pABCTE01

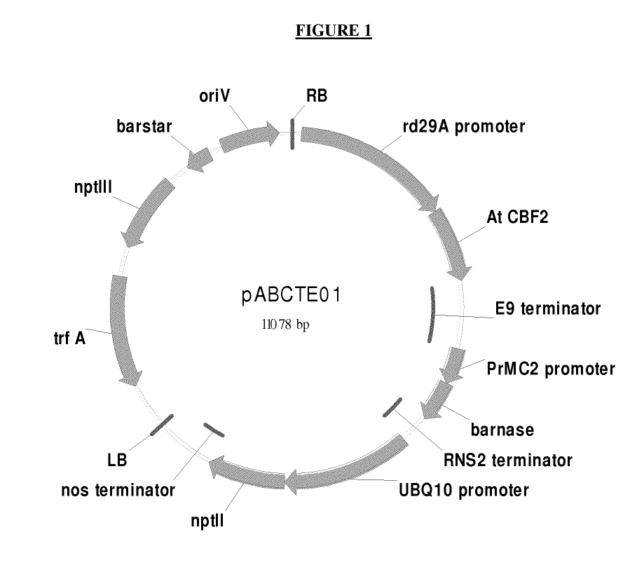

The plasmid pABCTE01 was introduced into hybrid variety EH1. As shown in FIG. 1, the vector has 11,078 base pairs and contains a CBF2 expression cassette, a barnase expression cassette, and an nptII selectable marker cassette between the left and right T-DNA border regions. The size of the T-DNA, between the right border (RB) and left border (LB), that is predicted to be incorporated into the Eucalyptus genome of transgenic lines is approximately 7.0 kb, and the remaining (unincorporated) backbone region of the plasmid is approximately 4.0 kb.

Provided below is a summary of vector pABCTE01 genetic elements, their position in the vector, and references for the source of these elements.

CBF2 Cassette

The CBF2 cassette is located within the T-DNA adjacent to the right border (RB) region. It consists of a cold-inducible promoter rd29A (Yamaguchi-Shinozaki and Shinozaki., 1993), the CBF2 (C-Repeat Binding Factor) cDNA, both from Arabidopsis thaliana, and the 3′ terminator region from the ribulose-1, 5-bisphosphate carboxylase subunit (RbcS2) from Pisum sativum (Coruzzi et al., 1984).

The CBF2 gene is part of the C-repeat/dehydration-responsive element binding factor (CBF/DREB) cold-response pathway (Jaglo-Ottosen et al., 1998; Zhang et al., 2004). Arabidopsis encodes a small family of cold-responsive transcriptional factors known as CBF], CBF2, and CBF3 (also called DREB1b, DREB1c and DREB1a, respectively). The CBF transcriptional factors belong to the AP2/EREBP family of DNA-binding proteins (Riechmann and Meyerowitz, 1998) and recognize the cold- and drought-responsive DNA regulatory sequence designated as C-repeat (CRT)/dehydration-responsive element (DRE), which has a conserved core sequence (Baker et al., 1994; Yamaguchi-Shinozaki and Shinozaki, 1994). This CRT/DRE core sequence was found to be present in the promoter regions of many cold-inducible genes including rd29A and cor15a (Maruyama et al., 2004) and it is believed that binding of CBFs to these promoters leads to increased expression.

It is known from the literature that overexpression of CBF genes under control of a constitutive promoter can increase cold tolerance but can also promote dwarfing (Zhang et al., 2004). To overcome this problem, stress-inducible plant promoters with a low background expression level have been used in conjunction with the cold tolerance genes (Yamaguchi-Shinozaki and Shinozaki, 1993). While any cold-inducible promoter may be used, vector pABCTE01 uses rd29A, a cold-inducible promoter isolated from Arabidopsis thaliana which confers induction of expression primarily under cold-stress conditions (Kasuga et al., 2004).

The phrase “CBF gene” refers to a gene that encodes a transcriptional activator that binds to the CRT (C-repeat)/DRE (dehydration responsive element) DNA regulatory element present in the promoters of many cold- and drought-inducible genes, including those designated COR (cold-regulated). The phrase “homologous CBF gene” refers to a gene that shares a high sequence identity or similarity with the CBF gene and has CBF function.

In this description, the phrases “CBF gene sequence” and “CBF homologous gene sequence” denote any nucleic acid, gene, polynucleotide, DNA, RNA, mRNA, or cDNA molecule that confers stress-related plant C-repeat binding factor (CBF) activity. Illustrative polynucleotides include but are not limited to the polynucleotides set forth in any of SEQ ID NOs: 1, 3, 5 or 7, or a sequence having at least 70%, 75%, 80%, 85%, 90%, 91%, 92%, 93%, 94%, 95%, 96%, 97%, 98%, 99% with any of SEQ ID NOs: 1, 3, 5 or 7.

A CBF or homologous CBF polynucleotide sequence may be identified from a myriad of plants characterized by the presence of a CBF gene. A CBF DNA sequence may be isolated as cDNA or genomic DNA from any suitable plant species using oligonucleotide primers or probes based on DNA or protein sequences disclosed herein or known within the art. Specific examples of plant species from which CBF genes may be isolated include dicotyledons, such as Cucurbitaceae, Solanaceae, Brassicaceae, Rutaceae, Papilionaceae, such as alfalfa and Vigna unguiculata, Malvaceae, Asteraceae, Malpighiaceae such as Populus, Myrtaceae such as Eucalyptus; and monocotyledons, such as gramineae, including wheat, barley, and corn. In one embodiment, a CBF gene is isolated from Arabidopsis thaliana, and a CBF homologous gene is isolated from Eucalyptus.

In this description, the phrases “CBF polynucleotide sequence” and “CBF homologous polynucleotide sequence” also refer to any nucleic acid molecule with a nucleotide sequence capable of hybridizing under stringent conditions with any of the sequences disclosed herein, and coding for a polypeptide with stress-related transcription factor activity equivalent to the polypeptides comprising amino acid sequences disclosed herein under SEQ Ill NOS: 2, 4,6 or 8. The phrases also include sequences which cross-hybridize with SEQ ID NO: 1, SEQ ID NO: 3, SEQ ID NO: 5, or SEQ ID NO: 7, which are at least 70% identical to the nucleotide sequence represented by SEQ ID NOs: 1, 3, 5 or 7. The nucleotide sequences of the invention may encode a polypeptide which is homologous to the polypeptides disclosed herein that comprise an amino acid sequence represented by SEQ ID NO: 2, SEQ ID NO: 4. SEQ ID NO: 6, or SEQ ID NO: 8.

“Stringent conditions” means conditions under which only base sequences coding for a polypeptide with stress-related transcription factor activity equivalent to the transcription factor encoded by a CBF gene sequence or CBF homologous gene sequence form hybrids with the specific CBF or CBF homologous sequence (referred to as specific hybrids), and base sequences coding for polypeptides with no such equivalent activity do not form hybrids with the specific sequence (referred to as non-specific hybrids). One with ordinary skill in the art can readily select such conditions by varying the temperature during the hybridization reaction and washing process, or the salt concentration during the hybridization reaction and washing process. Specific examples include but are not limited to conditions under which hybridization is brought about in 3.5.times SSC, 1.times.Denhardt's solution, 25 mM sodium phosphate buffer (pH 7.0), 0.5% SDS, and 2 mM EDTA for 18 hours at 65.degree. C., followed by 4 washes of the filter at 65.degree. C. for 20 minutes, in 2.times.SSC, 0.1% SDS, and a final wash for up to 20 minutes in 0.5.times.SSC, 0.1% SDS, or 0.3.times.SSC and 0.1% SDS for greater stringency, and 0.1.times.SSC, 0.1% SDS for even greater stringency. Other conditions may be substituted, as long as the degree of stringency in equal to that provided herein, using a 0.5.times.SSC final wash.

Additionally, CBF homologous gene sequences include fragments and variants of the polynucleotides represented by SEQ ID Nos: 1 or 3, 5 or 7, with one or more bases deleted, substituted, inserted, or added, that code for a polypeptide with stress-related transcription factor activity. The “base sequences with one or more bases deleted, substituted, inserted, or added” referred to here are widely known by those having ordinary skill in the art to retain physiological activity even when the amino acid sequence of a protein generally having that physiological activity has one or more amino acids substituted, deleted, inserted, or added. For example, the poly A tail or 5′ or 3′ end nontranslation regions may be deleted, and bases may be deleted to the extent that amino acids are deleted. Bases may also be substituted, as long as no frame shift results. Bases also may be “added”, as long as such modifications do not result in the loss of stress-related transcription factor activity. A modified DNA in this context can be obtained by modifying the DNA base sequences of the invention so that amino acids at specific sites are substituted, deleted, inserted, or added by site-specific mutagenesis, as described in Zoller & Smith, 1982, Nucleic Acid Res. 10: 6487-6500.

Terminator

The terminator for the cassette is from the 3′ untranslated region from the ribulose-1, 5-bisphosphate carboxylase subunit (RbcS2) isolated from Pisum sativum (Coruzzi et al., 1984).

Barnase Cassette

This cassette consists of a modified barnase gene from Bacillus amyloliquefaciens (Mossakowska et al., 1989, Meiering et al., 1992) under control of an anther-specific promoter (PrMC2) isolated from Pinus radiata as described in U.S. Application Publication No. 20030101487. The PrMC2 promoter was demonstrated to be active primarily in the tapetum of the pollen sac (Walden et al., 1999). Tissue specific expression of this promoter and efficacy in eliminating pollen production has been demonstrated in tobacco and other plant species.

Barnase in combination with the tapetum-specific TA29 promoter has been used previously to accomplish male sterility. Early experiments (unpublished results) suggested that even very low expression of barnase can be detrimental to the plant transformation and regeneration process. Applicants developed a modified form of the barnase gene with attenuated activity such that very low levels of expression would not impact overall plant development but would have sufficient activity to obtain ablation of developing pollen. The terminator for this cassette is the 3′ region from the RNS2 (Ribonuclease 2) gene from Arabidopsis thaliana (Taylor et al., 1993).

Selectable Marker Cassette

Neomycin phosphotransferase (nptII) from Escherichia coli transposon Tn5 was used as a selectable marker. The kanamycin resistance selectable marker gene used in this cassette is generally accepted as being safe (Fuchs et al., 1993) and used widely in several crop plants.

This cassette utilizes the Arabidopsis thaliana polyubiquitin (UBQ10) gene promoter (Norris et al., 1993). This promoter shows strong expression in a wide range of tissues and was selected based on its efficacy when driving nptII gene in plant transformation (ArborGen unpublished results). The terminator used for the nptII gene is from the nopaline synthase (nos) gene of Agrobacterium tumefaciens (Bevan et al., 1983).

T-DNA Borders

The right and left borders used in plasmid pABCTE01 were derived from the Ti plasmid of Agrobacterium tumefaciens strain C58. These sequences delineate the region of the plasmid to be transferred into the target plant genome and are required for efficient T-DNA transfer (Depicker et al., 1982; Barker et al., 1983).

Genetic Elements Outside the T-DNA Borders

Four elements are located in the vector backbone outside of the T-DNA borders, and therefore are not expected to be transferred into the Eucalyptus genome. These elements are necessary for bacterial maintenance and replication of the plasmid. The first element, trfA, is a bacterial origin of replication for plasmid maintenance in E. coli (Frisch et al., 1995). The second, nptIII, encodes a neomycin phosphotransferase gene conferring kanamycin resistance used in selecting for the vector in E. coli and Agrobacterium (Frisch et al., 1995). The barstar gene from Bacillus amyloliquefaciens has been used previously for bacterial plasmid maintenance when the barnase gene is present (Hartley, 1988, 1989). Finally, the oriV element is an origin of replication from pRK2 for plasmid maintenance in Agrobacterium (Stalker et al., 1981).

Illustrative Plants

The present disclosure comprehends the genetic manipulation of plants, to enhance their stress tolerance, by driving the expression of a CBF gene or a CBF homologous gene, preferably under the control of a promoter as described above. The result is enhanced freeze-tolerance.

The term “plant” denotes any fiber-containing plant material that can be genetically manipulated, including, but not limited to, differentiated or undifferentiated plant cells, protoplasts, whole plants, plant tissues, or plant organs, or any component of a plant such as a leaf, stem, root, bud, tuber, fruit, rhizome, or the like.

Exemplary plants include, but are not limited to, trees, such as Eucalyptus species and hybrids thereof (E. alba, E. albens, E. amplifolia, E. amygdalina, E. aromaphloia, E. baileyana, E. bailadoniensis, E. benjensis, E. benthamii, E. bicostata, E. botryoides, E. brachyandra, E. brassiana, E. brevistylis, E. brockwayi, E. calmaldulensis, E. ceracea, E. cloeziana, E. coccifera, E. cordata, E. cornuta, E. corticosa, E. crebra, E. croajingolensis, E. curtisii, E. dairympleana, E. deglupta, E. delegatensis, E. delicata, E. diversicolor, E. diversifolia, E. dives, E. dolichocarpa, E. dorrigoensis, E. dundasii, E. dunnii, E. elata, E. erythrocorys, E. erythrophloia, E. eudesmoides, E. falcata, E. gamophylla, E. glaucina, E. globulus, E. globulus subsp. bicostata, E. globulus subsp. globulus, E. gongylocarpa, E. grandis, E. grandis.times.urophylla, E. guilfbylei, E. gunnii, E. hallii, E. houseana, E. jacksonii, E. lansdowneana, E. latisinensis, E. leucophloia, E. leucoxylon, E. lockyeri, E. lucasii, E. macarthurii, E. maidenii, E. marginata, E. megacarpa, E. melliodora, E. michaeliana, E. microcorys, E. microtheca, E. muelleriana, E. nitens, E. nitida, E. obliqua, E. obtusiflora, E. occidentalis, E. optima, E. ovata, E. pachyphyila, E. pauciflora, E. pellita, E. perriniana, E. petiolaris, E. pilularis, E. piperita, E. platyphylla, E. polyanthemos, E. populnea, E. preissiana, E. pseudo globulus, E. pulchella, E. radiata, E. radiata subsp. radiata, E. regnans, E. risdonii, E. robertsonii, E. rodwayi, E. rubida, E. rubiginosa, E. saligna, E. salmonophloia, E. scoparia, E sieberi, E. spathulata, E. staeri, E. stoatei, E. tenuipes, E. tenuiramis, E. tereticornis, E. tetragona, E. tetrodonta, E. tindaliae, E. torquata, E. umbra, E. urophylla, E. vernicosa, E. viminalis, E. wandoo, E. wetarensis, E. willisii, E. willisii subsp. falciformis, E. willisii subsp. willisii, E. woodwardii); Populus species and hybrids thereof (P. alba, P. alba.times.P. grandidentata, P. alba.times.P. tremula, P. alba.times.P.tremula var. glandulosa, P. alba.times.P. tremuloides, P. balsamifera, P. balsamifera subsp. trichocarpa, P. balsamifera subsp. trichocarpa.times.P. deltoides, P. ciliata, P. deltoides, P. euphratica, P. euramericana, P. kitakamiensis, P. lasiocarpa, P. laurifolia, P. maximowiczii, P. maximowiczii.times.P. balsamifera sub.sp. trichocarpd P. nigra, P. sieboldii.times.P. grandidentata, P. suaveolens, P. szechuanica, P. tomentosa, P. tremula, P. tremula.times.P. tremuloides, P. tremuloides, P. wilsona, P. canadensis, P. yunnanensis); Conifers such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); cedars such as Western red cedar (Thuja plicata) and Alaska yellow-cedar (Chamaecyparis nootkatensis); citrus species, including C. medica, C. aurantifolia, C. latipes, C. limon; C. reticulata, C. sinensis, C. paradisi, C. aurantium, C. jambhiri, C. grandis, C. indica, C. ichangensis, C. tachibana, C. micrantha, and citrus hybrids, including, Palestine sweet lime, bergamot and Volkamer lemon, Rangpur lime and Rough lemon; avocado (Persea americana Mill), papaya (Carica papaya), nutmeg (Myristica insipida), pistachio (Pistacio vera), kiwi (Actinidia deliciosa A. Chev.), and jojoba (Simmondsia chinensis).

Fiber-producing plants also are included in this context. Illustrative crops are cotton (Gossipium spp.), flax (Linum usitatissimum), stinging nettle (Urtica dioica), hop (Humulus lupulus), lime trees (Tilia cordata, T. x. europaea and T. platyphyllus), spanish broom (Spartium junceum), ramie (Boehmeria nivea), paper mulberry (Broussonetya papyrifera). New Zealand flax (Phormium tenax), dogbane (Apocynum cannabinum), Iris species (I. douglasiana, I. macrosiphon and I. purdyi), milkweeds (Asclepia species), pineapple and banana.

The phrase “transgenic plant refers to a plant that has incorporated a DNA sequence, including, but not limited, to genes that are not normally present in a host plant genome, DNA sequences not normally transcribed into RNA or translated into a protein (”expressed“), or any other genes or DNA sequences normally present in the non-transformed plant, that are genetically engineered or have altered expression. The phrase “transgenic plant” encompasses primary transformants regenerated from calluses obtained from transformed plant cells (Rₒ plants), as well as their seed-derived R₁ and R2 progenies, and vegetatively-propagated derivatives of the plants and R_{0,} R₁ and R₂ progenies. The invention also contemplates production of hybrids using an R_{0,} R₁ and R₂ plant as a parent.

It is contemplated that, in some instances, the genome of a transgenic plant will have been augmented through the stable introduction of a transgene. In other instances, however, the introduced gene will replace an endogenous sequence. A preferred gene is a CBF gene or a CBF homologous gene. p For example, and non-limiting, Eucalyptus variety EH1, which is the progenitor of the freeze-tolerant line 427, was obtained from International Paper Co. in Brazil. This variety was identified as a hybrid between E. grundis and E. urophylla. EH1 was selected for its improved growth, superior wood quality and adaptability to different soil types and environments. These characteristics have made EH1 a preferred genotype for deployment in operational Eucalyptus plantations in Brazil. In the present disclosure, and in no way limiting, EH1 was used as a recipient variety for insertion of T-DNA to obtain freeze-tolerant lines.

Transformation Methodology

The present construct may be introduced into a host plant cell by standard procedures known in the art for introducing recombinant sequences into a target host cell. Such procedures include, but are not limited to, transfection, infection, transformation, natural uptake, electroporation, biolistics and Agrobacterium. Methods for introducing foreign genes into plants are known in the art and can be used to insert a construct into a plant host, including, biological and physical plant transformation protocols. See, for example, Miki et al., 1993, “Procedure for Introducing Foreign DNA Into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Borsch et al., Science 227:1229-31, 1985), electroporation, micro-injection, and biolistic bombardment.

Methods for transforming tree species are well known in the art. By no means limiting, explant refers to plant tissue that is a target for transformation and may include leaf, petiole, floral, and internodal tissues harvested from plants grown in vivo and/or in vitro. For example, a tree can be transformed by culturing a tree explant on a pre-culture medium before transformation. A pre-culture medium may comprise an Agrobacterium inducer, such as acetosyringone, as well as other plant growth regulators, such as auxin and/or cytokinin. Alternatively, other pre-culture media and time periods of culture may be used. Such methodology are known in the art and can be found in, e.g., U.S. application Ser. Nos. 10/981,742; 11/158,342; and 10/861,909.

The instant plant may be a direct transgenic, meaning that the vector was introduced directly into the plant, such as through Agrobacterium, or the plant may be obtained by asexual reproduction of a transgenic plant.

Measuring Freeze Tolerance

Cold tolerance can be assessed in numerous ways, including both quantitative measurements as well as observational differences between a control plant and a genetically modified plant. For example, and in no way limiting, freeze tolerant phenotype could include, e.g., increased height, less leaf damage, less dieback of main stem, less crown defoliation, and/or improved tree stem form (single dominant leader stem). Thus, for example, compared with a control plant, a freeze-tolerant plant may display increased height, less leaf damage, less dieback of main stem, less crown defoliation, and improved tree stem form (single dominant leader stem).

As shown in the Examples, for instance, illustrative Eucalyptus FTE-427 displays freeze tolerance down to approximately 18 degrees F., and has increased height, less leaf damage, less dieback of main stem, less crown defoliation, and/or improved tree stem form (single dominant leader stem) compared with a control plant. The crown score data was based on visual observation of leaf defoliation on a scale of 0 to 100 (0=complete defoliation; 100=complete canopy retention) and dieback was calculated as the percent difference in live height at the end of winter compared to pre-winter height measurements.

Illustrative Products

Contemplated herein are methods for obtaining wood, wood pulp, paper, tea, and oil from a plant transformed and selected by the methods disclosed herein. Methods for transforming and selecting a transgenic plant are provided above and are well-known in the art. A transformed plant can be cultured or grown under any suitable conditions. For example, Eucalyptus can be cultured and grown as in, for example, Rydelius, et al., Growing Eucalyptus for Pulp and Energy, presented at the Mechanization in Short Rotation, Intensive Culture Forestry Conference, Mobile, Ala., 1994. Wood, wood pulp, paper, and oil can be obtained from the plant by any means known in the art.

As noted above, the wood and wood pulp obtained herein may demonstrate improved characteristics including, but not limited to any one or more of lignin composition, lignin structure, wood composition, cellulose polymerization, fiber dimensions, ratio of fibers to other plant components, plant cell division, plant cell development, number of cells per unit area, cell size, cell shape, cell wall composition, rate of wood formation, aesthetic appearance of wood, formation of stem defects, rate of growth, rate of root formation ratio of root to branch vegetative development, leaf area index, and leaf shape include increased or decreased lignin content, increased accessibility of lignin to chemical treatments, improved reactivity of lignin, increased or decreased cellulose content increased dimensional stability, increased tensile strength, increased shear strength, increased compression strength, increased shock resistance, increased stiffness, increased or decreased hardness, decreased spirality, decreased shrinkage, and differences in weight, density, and specific gravity.

The following examples are illustrative and non-limiting. It should be understood that many variations and modifications can be made while remaining within the spirit and scope of the disclosure.

### EXAMPLE 1

### Plant Materials

The Eucalyptus variety EH1, which is the progenitor of the FTE-427, was obtained from International Paper Co. in Brazil. This variety was identified as a hybrid between E. grandis and E. urophylla. EH1 was selected for its improved growth, superior wood quality and adaptability to different soil types and environments. These characteristics have made EH1 a preferred genotype for deployment in operational Eucalyptus plantations in Brazil. EH1 was used as a recipient variety for insertion of T-DNA to obtain freeze-tolerant lines.

The sterile tissue culture shoots of EH1 were transferred from Brazil to ArborGen's contract research laboratories (Trees and Technology/Horizon 2, TeTeko, NZ) in New Zealand. The shoot cultures were micropropagated and maintained on solid MS medium (Murashige and Skoog, 1962) supplemented with 1 μM BAP and 20 g/L sucrose. Shoot cultures were transferred to fresh medium every 3-4 weeks and grown in a growth chamber at 25±2° C. under a 16-hour photoperiod and low light intensity provided by cool white fluorescent tubes.

### EXAMPLE 2

### Agrobacterium Preparation and Transformation

Agrobacterium tumefaciens strain EHA105 (Hood, 1993; McBride and Summerfelt, 1990) harboring construct pABCTE01 was used for transformation.

Agrobacterium tumefaciens cultures were initiated from frozen glycerol stocks (500 in 10 ml YEP broth (Lichtenstein and Draper, 1986) supplemented with 50 mg/L kanamycin and 50 mg/L rifampicin. The culture was grown overnight at 25° C. on an orbital shaker (200 rpm), pelleted by centrifugation at 3000×g for 10 minutes and resuspended in 20-30 ml liquid MS (2.0% w/v glucose, no plant growth regulators or antibiotics) for explant inoculation.

Leaf explants of EH1 were harvested from actively growing micropropagated shoot clumps, inoculated with the resuspended Agrobacterium cells and plated on MS-based co-cultivation medium as described by Cheah (2001). The explants were co-cultivated for 4 days under low light at approximately 22° C. in a growth chamber.

Following co-cultivation, explants were transferred to regeneration medium (Cheah, 2001) containing 50 mg/L kanamycin to allow selection of transformed cells and 250 mg/L timentin to kill any remaining Agrobacterium. After two to three weeks, shoot primordia were produced at the base of leaf explants. The developing shoot primordia were transferred to the same basal regeneration medium containing 100 mg/L kanamycin. Four weeks later, the shoot primordia converted into adventitious shoots that were then maintained for 12 weeks on selection medium containing 150 mg/L kanamycin by subculturing at 4 week intervals. Individual kanamycin resistant shoots were recovered from each event (designated as a transgenic line) at 16 to 20 weeks after co-cultivation. From each actively growing putative transgenic shoot, two to three young leaves were harvested for molecular verification. DNA was extracted from leaf samples and analyzed by PCR using standard procedures for the presence of genes-of-interest, selectable marker gene and the absence of vector backbone, as well as for insert copy number.

### EXAMPLE 3

### Shoot Propagation and Rooting of Transgenic Lines

Shoot cultures were maintained and identity-preserved for each confirmed transgenic line on MS-based medium containing 50 mg/L kanamycin and 250mg/L timentin by subculturing every 4 weeks. The antibiotics were eliminated from the medium at shoot elongation. For shoot elongation and root induction, the elongated shoots of the confirmed transgenic lines were harvested and placed on JADS medium (Vanderlei, 2002).

The sterile rooted tissue culture plants or shoot cultures of transgenic lines and non-transgenic control plants of the same parental genotype produced in New Zealand were imported into the US under approved BRS import permits. Upon arrival in the US, the individual rooted plants of transgenic lines were transferred to soil in suitable containers, labeled appropriately using a durable water insoluble label, and grown in our secure greenhouse facilities in South Carolina. The transgenic plants were then acclimatized outdoor and field tested under acknowledged BRS notifications and permits.

### EXAMPLE 4

### Molecular Characterization of FTE 427

Molecular analysis was performed on freeze-tolerant Eucalyptus line 427 to characterize the integrated T-DNA. Southern blot analysis was used to determine insert number, copy number, cassette intactness and to confirm the absence of vector backbone.

In vitro leaf tissue was harvested from replicated shoot cultures grown in a growth chamber. Control leaf samples were obtained from untransformed shoot cultures of the hybrid Eucalyptus variety used for transformation (EH1). Leaf tissue was harvested periodically from the in vitro shoot cultures throughout the study.

Plasmid pABCTE01, used in the production of line 427, also served as a reference substance. For Southern blot analyses, standards and positive hybridization controls were created using specific quantities of plasmid pABCTE01 spiked into Calf Thymus (Sigma, Cat. No. D4764) carrier DNA which was then digested with designated restriction enzymes. The amount of spiked pABCTE01 plasmid (60 pg) representing a single copy per diploid genome was calculated based on the formula:

# pg =(M*10⁶*P)/G

where M=# micrograms of genomic DNA run in a lane, 10⁶=conversion from μg to pg, P=size of plasmid in bp, G=size of diploid genome in bp. The calculation used 11078 by for pABCTE01, a diploid genome size of 1.33×10⁹ base pairs (Grattapaglia and Bradshaw, 1994), and 7 micrograms of genomic DNA. A molecular size marker (New England Biolabs, Cat. No. N3232L, 10 kb-0.5 kb) was used for size estimations on Southern blots. In the following discussion, a single copy per diploid genome is referred to as the 0.5 copy standard to reflect the amount of spiked DNA on a haploid genome basis.

### Purification of Genomic DNA

DNA from both transgenic and untransformed samples was purified using a CTAB extraction protocol. Two grams of in vitro leaf material were added to a mortar and ground into a fine powder in the presence of liquid nitrogen. The powder was placed into a labeled 35 ml Oakridge style tube containing 14 ml of CTAB extraction buffer (0.1 M iris pH 7.5, 0.7 M NaCl, 10 mM EDTA, 1% CTAB, 1% PVP), the tube was sealed and then incubated at 60° C. for 15 min. with periodic agitation. Cellular debris was pelleted by centrifugation at ˜10000×g for 5 min. The supernatant was poured into a second labeled 35 ml Oakridge style tube containing 14 ml of phenol (Sigma, Cat. No. P4557, pH 10.5) and inverted several times to create a homogenous emulsion. The emulsion was separated into two phases by centrifugation at 14000×g for 5 minutes. The upper aqueous layer was removed and added to a fresh tube containing 14 ml of chloroform/isoamyl alcohol (Sigma, Cat. No. 00549, 24:1). The tube was agitated for several minutes to form a uniform emulsion followed by centrifugation at 14000×g for 5 min. The aqueous layer was removed and added to a fresh tube containing 14 ml of chloroform/isoamyl alcohol (24:1) with 10% CTAB (0.1 M Tris pH 7.5, 0.7 M NaCl, 10 mM EDTA, 10% CTAB, 1% PVP). The tube was inverted several times again followed by centrifugation at 14000×g for 5 min. The aqueous layer was removed and placed into a newly labeled 35 ml Oakridge style tube and combined with 8 ml of 3 M NaOAc (pH 4.8) followed by 9 ml of isopropanol. The tube was then gently inverted several times. The DNA was pelleted by centrifugation at 14000×g for 20 min., rinsed once with 70% ethanol and air dried for up to 1 hour. The DNA was then resuspended in TE buffer (10 mM Tris-Cl, pH 7.5, 1 mM EDTA).

DNA samples were quantitated using a SpectraMAX Gemini Fluorescence microplate reader (Molecular Devices, Inc.) using standards of known concentration (1 kb DNA ladder, New England Biolabs) for calibration.

### Restriction Endonuclease Digestion

Digest reactions for untransformed control samples contained 7 μg of genomic DNA and were performed overnight at 37° C. in a total volume of 400 μl using 50-100 units of the appropriate restriction enzyme. For the translines, samples were prepared for both a long run and a short run on the electrophoresis gels by digesting a total of 14 μg genomic DNA in 800 μl in the same reaction overnight at 37° C. This digest was then separated equally into two tubes (7 μg each) and precipitated. Whole plasmid pABCTE01, used as a positive hybridization control, was spiked into 7 μg of calf thymus DNA prior to incubation. Following digestion, samples were precipitated by adding 40 μl of 3M NaOAc, pH 5.2 and 0.7 volumes of isopropanol. The DNA was pelleted by centrifugation at 14000×g for 10 minutes, washed briefly with 70% ethanol, briefly air dried and resuspended in 60 μl of TE buffer. To facilitate gel loading, samples were loaded into a speedvac and spun for 40 minutes to reduce the overall volume and to remove residual ethanol.

### DNA Probe Preparation for Southern Blot Analysis

Template DNA for hybridization probes was prepared by either restriction endonuclease digestion or PCR amplification of purified plasmid pABCTE01 (FIG. 1). In both cases, following completion of the reaction, samples were run on an agarose gel and the appropriate band was purified using a commercially available kit (Qiagen, Cat. No. 28604). Approximately 25 ng of each probe template was labeled with α ³²P-dATP using a random priming reaction (Invitrogen Inc., Cat. No. 18187-013) as described by the manufacturer. Radiolabeled probes were purified using column chromatography (BioRad, Cat. No. 732-6231).

### Southern Blot Methods

DNA samples were analyzed using standard Southern blot analysis (Southern, 1975) by digesting samples with restriction endonucleases and separating the resulting fragments by electrophoresis on 0.8% agarose gels that were run in 1× TAE buffer (40 mM Tris-acetate pH 8.3, 1 mM EDTA). Two runs were performed for each sample on each gel. A long run enabled greater resolution of high molecular weight fragments while a short run allowed the observation of low molecular weight fragments. The long run samples were loaded onto the gel and run overnight at 20V. Short run samples were loaded the next day in lanes adjacent to the long run samples and run at 140V for 2 hours. A molecular size marker (New England Biolabs, Cat. No. N3232L, 10 kb-0.5 kb) was used for size estimations on each run. Following electrophoresis, gels were stained with ethidium bromide for 10 minutes, destained for 10 minutes and then photographed.

The gels were placed into a depurination solution (0.125 N HCL) and gently rocked for 12 minutes followed by denaturing solution (0.5 M NaOH, 1.5 M NaCl) for 30 minutes and then a neutralizing solution (0.5 M Tris-HCL pH 7.0, 1.5 M NaCl) for 30 minutes. DNA was transferred to Zeta-Probe nylon membranes (BioRad, Cat. No.162-0165) overnight using 20×SSC (3 M NaCl and 0.3 M sodium citrate, pH 7.0) using standard Southern blotting techniques (Southern, 1975). The following day, blots were covalently crosslinked to the membrane using the “autolink” setting on a UV Stratalinker (Stratagene) and then oven dried at 65° C. for 20 minutes. Blots were prehybridized for 1-2 hours using a hybridization solution containing 0.25 M sodium phosphate pH 7.2 and 7% SDS. Probe was added directly to the prehybridization solution and allowed to hybridize for 16-20 hours at 65° C. Membranes were washed three times using 0.1% SDS and 0.1×SSC for 20 minutes at 65° C. Multiple exposures were obtained using Kodak BioMax MS film with two intensifying screens at −80° C. Typical exposure times were 2-3 days.

### Insert Number Analysis

The number of inserts (number of insertion sites within the genome) was analyzed by digesting DNA from each transline with three restriction endonucleases (Age I, ApaL I and Nhe I) concurrently, none of which cut within plasmid pABCTE01. This restriction digest would release an intact T-DNA flanked on either side by a portion of plant genomic DNA. After hybridization with a T-DNA-specific probe, the number of observed bands would be indicative of the number of T-DNA inserts present within the genome: lines containing a single insert would be indicated by a single band. The size of the fragment is a function of the restriction sites in the flanking plant DNA, thus multiple inserts would be expected to yield different size fragments.

### EXAMPLE 5

### Field Trials for Phenotypic Characterization of FTE-427

Field Trials were established at multiple sites where the trees would he subjected to different levels of freeze-stress based on historic weather patterns. At each location, and for each experimental trial, preparations and tasks prior to and directly after planting were targeted at optimizing plant survival and productivity. For different locations the methods used in establishing trials were tailored to local conditions including the suitability and availability of equipment and methods used to manage prior existing vegetation at the site. Almost all sites were irrigated immediately after planting, followed by periodic irrigation for several weeks to ensure good survival and establishment.

Field experiments established in freeze-stress environments were initially evaluated for a number of different phenotypes that were indicative of improved freeze tolerance. The phenotypic observations included tip and leaf damage following specific freeze events. A waiting period of several days was required for any phenotype to manifest itself following the freeze event. In many cases this was confounded by subsequent freeze events and it was difficult to specifically assign a given observation to a defined time point and temperature. Nevertheless, the data from these initial observations were important in allowing the selection of a few potential candidate lines that merited further testing, including FTE-427.

Following these initial observations it was concluded that a simple comparison of pre-winter and post-winter height measurements, used to calculate a percent dieback of the main stem, together with a post-winter assessment of defoliation based on a qualitative observation of leaf retention (crown score) provided appropriate assessment of freeze tolerance phenotype and field performance. These measurements also avoided the challenge of mid-winter assessments that could be complicated by overlapping and incremental freeze events. Post-winter survival was also assessed. For trees that were killed to or near ground level survival was judged based on the observation of new shoots emerging from the killed stem near ground level.

A simple temperature recorder (HOBO Outdoor 4 Channel, Onset Computer Corporation) was used to obtain data on freeze events with temperatures recorded at 15 minute intervals. At sites where there was no on-site recorder or a mechanical failure of the recording device occurred (for example rodents chewed through wires at one site) the temperature data were obtained from the nearest available public source(s). Temperature data were used to determine the absolute minimum temperature and, when available, for calculating cumulative hours at or below defined temperature thresholds (25, 20 and 15° F.).

### EXAMPLE 6

### Field Test Results from Baldwin County, Alabama

This site represents a location typical of the USDA Hardiness Zone 8b where freeze-tolerant Eucalyptus FTE-427 is most likely to be grown.

Based on typical weather patterns observed at this site, it was anticipated that in mild winters there would likely be minimal damage to both FTE-427 and the EH1 controls while in more severe winters there would be a clear differentiation between them. Average annual precipitation at this site is 67.9 inches (1725 mm). The soil type is a Magnolia fine sandy loam with little to no slope. The trial consisted of a randomized complete block design with single tree plots (48 lines in total) with eight replicates. The trial was planted on Nov. 8, 2005 and the area covered by the site was ˜1.1 acres. The test was irrigated immediately after planting and then periodically over the next several weeks to ensure good establishment.

### 1) Growth and Freeze Tolerance Assessment in 2005 and 2006

Trees were established before the first freeze event (30.1° F.) that occurred on the night of November 18, 2005. No significant damage was observed on control and transgenic trees from this freeze event because our data from cold chamber experiments suggests that EH1 control trees have a natural freeze tolerance down to ˜26° F.).

At this site, only three freeze events occurred that experienced temperatures below 25° F. (24.2° F. on Dec. 22, 2005; 23.3° F. on Jan. 7, 2006; and 21.6° F. on Feb. 12, 2006). Assessment of damage to growing tips on the trees from the December 22nd, 24.2° F. freeze event showed 98% of tips damaged for EH1, but 0% for 427. Heights were measured in early January (Jan. 6, 2006) and at that time EH1 was significantly taller than FTE-427. This may be a reflection of differences in the size of plants at establishment rather than any growth or freeze damage.

A second assessment of tip damage made on Feb. 20, 2006, eight days after the freeze event (21.6° F.), showed that freeze damage to growing tips was higher for EH1 control trees (58.1%) compared to FTE-427 (12-20%) but the difference was not statistically significant. A canopy injury assessment at that time did not show any trends. Leaf damage scores (Leaf damage scores were rated as: 1=<5% leaf damage; 2=5-50%; 3=50-90%; 4=>90% leaves damaged) assessed two weeks later in early March did show real trends. At this time point 90% of EH1 trees had a leaf damage score of 3 (50-99% leaf damage) whereas the trees of FTE-427 had a leaf damage score of 2 (5-50% leaf damage) or better, indicating increased freeze tolerance in transgenic trees compared to control trees.

TABLE 1
Mid-winter assessment of freeze damage in 2005/2006 Baldwin County
field study. Leaf damage scores were rated as: 1 = <5% leaf damage;
2 = 5-50%; 3 = 50-90%; 4 = >90% leaves damaged
Percentage of Trees within Each Leaf Damage Score Class

Score

| | 1 (<5%) | 2 (5-50%) | 3 (50-90%) | 4 (>90%) |
|---|---|---|---|---|
| EH1 | 0.0% | 9.4% | 90.6% | 0.0% |
| FTE- | 62.5% | 37.5% | 0.0% | 0.0% |
| 427 | | | | |

Although the above observations were somewhat confounded by overlapping freeze events, there was sufficient differentiation between e FTE-427 and the EH1 control to indicate that that FTE-427 demonstrated improved freeze tolerance.

At the end of winter, dieback of the main stem was assessed. Dieback was calculated as the percent difference in live height at the end of winter compared to pre-winter height measurements. Live height was measured from ground level to the point at which new spring growth emerged on the main or dominant stem. For this test, live height was measured in March 2006 and percent dieback was calculated for each tree. Control and transgenic trees were not significantly different for percent dieback and showed dieback percentages in the 5-7% range. As shown in Table 2 (March 2006 measurement), the average live height of EH1 (1.34 feet) was significantly greater (99% level) than that of FTE-427 (1.12 feet). Survival (as measured in November 2006) was not significantly different for the control and transgenic trees.

TABLE 2
Phenotypic Comparisons between Control EH1 and FTE-427 obtained
from the Baldwin County 2005/2006 Field Study

| Trait | EH1 | 427 |
|---|---|---|
| Height (ft) Jan. 6, 2006 | 1.42 | 1.18** |
| Trees with Tip Dieback % Jan. 6, 2006 | 93.8 | 0.0 |
| Canopy Injury % Feb. 20, 2006 | 14.2 | 20.5 |
| Apical Damage % Feb. 20, 2006 | 58.1 | 19.7 |
| Height (ft) March 2006 | 1.34 | 1.12** |
| Dieback % March 2006 | 5.3 | 5.2 |
| Height (ft) May 2006 | 3.79 | 3.09* |
| Height (ft) November 2006 | 19.8 | 19.1 |
| Height Growth (ft) 2006 | 18.4 | 17.9 |
| DBH (in) November 2006 | 2.07 | 2.01 |
| Volume Index (ft³) November 2006 | 0.61 | 0.54 |
| Survival (%) November 2006 | 96.9 | 100 |
| Live Height (ft) April 2007 | 17.9 | 16.8 |
| Stem Dieback (%) April 2007 | 9.6 | 12.1 |
| Crown Score April 2007 | 78 | 79 |
| Survival (%) December 2007 | 96.9 | 100.0 |
| Live Height (ft) December 2007 | 38.2 | 37.1 |
| DBH (In.) December 2007 | 4.14 | 4.02 |
| Volume Index (ft³) December 2007 | 4.61 | 4.19 |
| 2007 Height Growth | 18.4 | 18.0 |
| 2007 DBH Growth | 2.07 | 2.01 |
| 2007 Volume Index Growth | 3.99 | 3.65 |
| Survival (%) April 2008 | 96.9 | 100.0 |
| Live Height (ft) April 2008 | 3.9 | 33.8** |
| Stem Dieback (%) April 2008 | 89.9 | 9.0** |
| Crown Score April 2008 | 0 | 55** |

Two parameters were deemed to best assess the performance of FTE-427 versus the EH1 control. Pre- and post-winter height measurements were used to calculate percent dieback for each tree as an indicator of freeze tolerance. Assessment of crown defoliation at the end of the winter also proved to be a good indicator of freeze damage. Since these parameters proved to be consistent and reliable indicators of freeze damage they were used in all subsequent trials.

### (2) Growth Assessment in the 2006 Growing Season for the Baldwin County Field Study

Tree stem form was affected by the freeze damage to apical growing tip. Therefore, all trees in this test were observed twice in 2006 for tree stem form. The mid-year (6/06) assessment showed that all trees of FTE-427 had single dominant leader stem characterizing normal tree stem form. In contrast, only 3% of the EH1 control trees had normal stem form. The remaining control trees showed one or more stems emerging from a lateral bud. Since EH1 has strong apical dominance, the year-end assessments showed that all but ˜13% of the EH1 trees had a single dominant leader stem. However, all trees of FTE-427 showed a single dominant leader stem form at the end of the year assessment suggesting that tree stem form was not affected in FTE-427.

Growth measurements taken in May 2006 showed that average height of FTE-427 was significantly shorter than EH1. However, by November 2006 the average height and overall growth of FTE-427 was not significantly different from EH1 control trees. In contrast to the height measurements taken in November 2006, FTE-427 was not significantly different from EH1 control trees for DBH, tree volume index and overall survival of the trees. Notably, in the 2007 growing season, FTE-427 showed non-significant differences in height and growth increments compared to EH1 control trees. These observations suggest that despite small seasonal variations noted in the growth of FTE-427, its growth was comparable to control trees for growth parameters before they were subject to a significant freeze event.

### 3) Freeze Tolerance Assessment in 2006/2007 Baldwin County Field Study

The lowest recorded temperature for the 2006/07 winter at this site was 20.6° F. on Feb. 16, 2007. Temperatures below 25° F. were also recorded on three other dates (23.3° F. on Dec. 8, 2006; 22.4° F. on Dec. 9, 2006; and 22.4° F. on Feb. 18, 2007).

As discussed above, the freeze damage for 2006/07 winter season was assessed using the crown score and stem dieback observations. The crown score data was based on visual observation of leaf defoliation on a scale of 0 to 100 (0=complete defoliation; 100=complete canopy retention) and dieback was calculated as the percent difference in live height at the end of winter compared to pre-winter height measurements. For 2006/07 winter season, the crown score and percent dieback observations for FTE-427 and control trees did not show significant differences. These observations indicated that it was not possible to discriminate between the FTE-427 and the EH1 controls in terms of freeze tolerance at this site for 2006/07 winter season.

### 4) Growth Assessment in 2007

FTE Line 427 was comparable to EH1 control trees with respect to average height, overall growth, diameter at breast height (DBH) and volume index (height×DBH²) measurements.

### 5) Freeze Tolerance Assessment in 2007/2008

The lowest recorded temperature at this site during the 2007/08 winter was 19.7° F. on the night of Jan. 2, 2008. Despite being less than one degree colder compared to the low temperature of 20.6° F. recorded in 2006/07 winter, there was a dramatic difference in the freeze damage between FTE-427 and control trees at this site in 2007/08.

At the end of the 2007/08 winter season, the crown score for EH1 was 0%, indicating that all trees were totally defoliated. In contrast, the FTE-427 had crown scores of 55.6%. EH1 control trees also showed dramatic dieback, with an average of 89.9% of the tree stem being killed as opposed to only 9% dieback observed for FTE-427. The post winter average live height for EH1 control trees was only 3.9 feet. The average live height for FTE-427 was over 33 feet, almost 10 fold higher than for EH1. These differences in crown score, dieback and live height between FTE-427 and EH1 control trees were highly significant.

It is likely that the notable differences observed in freeze damage during the two winter seasons may have resulted from the very different weather patterns experienced at this site. Temperatures prior to the 19.7° F. low of 2007/08 had been mild, with only four other periods (a total of 14.25 hours) at which the temperature fell below 32° F., the lowest of these being 28.5° F. In contrast, the lowest temperature in the 2006/07 winter was preceded by 28 separate freeze temperature periods (totaling over 150 hours), including several nights with temperature in the mid 20° F. Based on the literature, it is known that in freeze-tolerant plants, the freeze tolerance response is induced at low but non-damaging temperatures. We therefore speculate that the repeated induction of freeze tolerance response at low but non-damaging temperatures in early to mid winter of 2006/07 provided a good amount of protection for both the FTE-427 and EH1 when the temperature dropped to 20.6° F. In the relatively milder winter of 2007/08, there were fewer cold periods under which a freezing response would likely have been induced. Under these conditions EH1 was not able to tolerate a temperature drop to 19.7° F. whereas FTE-427 expressing CBF from the cold-induced rd29A promoter were able to withstand this temperature.

These observations demonstrate that the desired freeze tolerance phenotype was achieved in FTE-427 which contains the pABCTE01 construct. The data also suggests that freeze tolerance phenotype is capable of providing protection to Eucalyptus hybrid trees under variable and often dramatic temperature fluctuations commonly experienced during winter months in the southeastern US.

The data collected over three growing seasons clearly showed that FTE-427 grew similarly to the control trees in non-cold challenged conditions, but that FTE-427 can survive and grow after experiencing cold temperatures commonly occurring in the southeastern U.S. which significantly damaged or killed the non-transgenic control. The data highlights the importance of collecting observations for more than one growing/winter season to effectively evaluate freeze-tolerant phenotype as the variables that contribute to freeze-stress are wholly dependant on prevailing and variable natural conditions. The data also point to some of the challenges that are created by the subtle differences in weather patterns from year to year (and site to site). The apparent one degree difference in minimum low temperature between the two winter seasons was likely modulated by other factors that could include differences in wind speed, rainfall and soil moisture, rate of temperature change, and temperature patterns in the days (or weeks) preceding the freeze event. This does not undermine the importance of minimum temperature as a meaningful measure of freeze tolerance. It simply points to the difficulties of making predictive calls based on temperature alone since for any given temperature a multitude of other dynamic environmental factors will also impact freezing effects. A key consideration therefore is that the modified freeze-tolerant trait should be able to provide protection against normal temperature fluctuations expected in a given region over several growing seasons and it should not be tied to an absolute tolerance to a fixed minimum temperature.

There was a very high negative correlation (−0.87) between minimum temperature and dieback in EH1. For FTE-427 dieback was most highly correlated with cumulative hours at or below 25° F. (data not shown). Cumulative freeze hours were calculated based on temperatures below different set points of 32, 25, 20 and 15° F. In growth chamber tests, the highest temperature for which a 24 hour exposure gave visible signs of leaf damage in EH1 but not in FTE-427 was 25° F.

What is claimed is:

1. A transgenic Eucalyptus displaying a freeze-tolerant phenotype.

2. The transgenic Eucalyptus of claim 1, wherein said plant is a hybrid.

3. The transgenic hybrid of claim 2, wherein said hybrid has a genotype of E. grandis×E. urophylla.

4. The transgenic Eucalyptus of claim 1, wherein said freeze-tolerant phenotype is any one or combination of increased height, less leaf damage, less dieback of main stem, less crown defoliation, and/or improved tree stem form (single dominant leader stem), relative to a control plant.

5. Wood or wood fiber produced from transgenic Eucalyptus of claim 1.

6. Pulp produced from the transgenic Eucalyptus of claim 1.

7. Paper produced from the transgenic Eucalyptus of claim 1.

8. Oil produced from the transgenic Eucalyptus of claim 1.

9. A method of conferring freezing tolerance in Eucalyptus, comprising
(a) introducing into a Eucalyptus cell a construct comprising an isolated polynucleotide sequence operably linked to a promoter, wherein said isolated polynucleotide encodes a CBF polypeptide and wherein the promoter drives the expression of the CBF polypeptide Eucalyptus upon exposure to cold for a period of time;
(b) culturing the transformed Eucalyptus cell under conditions promoting growth of a transgenic Eucalyptus tree, thereby producing a transgenic tree;
(c) subjecting the transgenic Eucalyptus tree to cold acclimation;
(d) exposing the transgenic tree to a freezing temperature for a period of time; and
(e) allowing the transgenic tree to recover under conditions that promote growth of the transgenic tree.

10. The method of claim 9, wherein said promoter is Arabidopsis thaliana rd29A promoter.

11. The method of claim 9, wherein said freezing temperature is from about 0° C. to about −30° C.

12. The method of claim 9, wherein said period of time is from about 2 hours to about 72 hours.

13. The method of claim 9, wherein said isolated polynucleotide sequence is selected from the group consisting of SEQ ID NO: 1, SEQ ID NO: 3, SEQ ID NO: 5 and SEQ ID NO: 7, or a fragment or variant thereof having at least 70% sequence identity to SEQ ID NO: 1, SEQ ID NO: 3, SEQ ID NO: 5, or SEQ ID NO: 7.


SEQUENCE LISTING (9 sequences)

SEQ ID NO: 1 - DNA, 826 bp, Eucalyptus sp.
atggacttctccgacaaggaagtgcagctcgcgtccgaccacccgaagaagcccgccgggaggaagaagttccgggagacccgccaccccgtgtaccgcggggtgcgtctgcgcgactcgggcaagtgggtctgcgaggttcgcgagcccaaaaagaagtcgaggatctggctcggcaccttccctactgtggagatggcagcgagggcgcatgacgtggcagcgctcgagctgagaggccagtctgcctgcctcaacttcgcagactctgcgtggcggctgcccaagccggcatcggcggatcccaaggacatacagaaggcggcggccaaggctgcagaggcattccagccgctggaatccgaggacataatgtcaggggacgagaagaagctgcattcggaagagggagtgtcgtacgacgaggaggacatcttggggatgccagggttgatcgccgatatggcggaggggatgctcttgtctcccccacaatgcggcgaagatatatatggcggagaggacgacgggaacttggacgcatacgcgtcattatggagccattccatctgaccatttctcaatttcacttgtacatagaagctgtttaatagagtgcggaagccactttttgtgatgtgccattagataaagagcatatcgatgaagcacgggtcttccgatgttgggccgccgatagagcggatgggaaagtgaaacaaagcgcaaaagtaggctgcgttcacagcaacatcgggcgttgtttatagttaataaagaaaccgcccgagccatcatgaatttgagagaagtggttttcaaactt

SEQ ID NO: 2 - protein, 190 aa, Eucalyptus sp.
MDFSDKEVQLASDHPKKPAGRKKFRETRHPVYRGVRLRDSGKWVCEVREPKKKSRIWLGTFPTVEMAARAHDVAALELRGQSACLNFADSAWRLPKPASADPKDIQKAAAKAAEAFQPLESEDIMSGDEKKLHSEEGVSYDEEDILGMPGLIADMAEGMLLSPPQCGEDIYGGEDDGNLDAYASLWSHSI

SEQ ID NO: 3 - DNA, 989 bp, Eucalyptus sp.
atggactcttcctcttatatctcccaccccaactcctttagcttcgacttcgccgagccgcctttctcgttgctgctgtcccacgaccggagcgcagcgcccgggaacttctctggggaggaggtgcgcctcgcgtccgaccacccgaagaagcccgccggcaggaagaagttccgggagacacgccaccccgtgtaccgtggggtgcggctgcgcgactcgggaaagtgggtctgcgaggtccgcgaacccagaaagaagtcgaggatctggcttggcaccttccccaccgcagacatggcagcgagggcgcacgatgtggcggcgctcgcactaaggggccagtccgcctgcctcaacttcgcagactccgcgtggcggctgccggtgccggcgtcacccaacaccaaagacatacagaaggcagcaaccaaggccgcggaggcattccagctgctggaatccgaggatgtgatgtcggggaacgagaagaagttgcattcggaagagggagtgttgtacgacgaggaggacatcttcgggatgccggggttgctcgccaatatggcggaggggatgctcttgtctccaccggaatgcagcggagatttatatgccggactggacgacgggaacttggacgcatacgcgtcattatggagctattccatgtgaccatttctcaatttcactttaagtgttttgtcattagggacgactttgatgccccacttagtaagtgttttaatagagtgcggataaaagttgaagcgtatgaacacagatattttcggaagccatttttgtggagtgccattagataaaaaggctgtgttcaccgcaacatcgggcgtttgcatatagttaataaagaaactgcccgagtcaacatgaatttaagagaagtggttttcagacttacccaatttatcttcgaatgtatcatgtctatggcaaaggtgcgccaacgaaattcgtatgggttcttt

SEQ ID NO: 4 - protein, 224 aa, Eucalyptus sp.
MDSSSYISHPNSFSFDFAEPPFSLLLSHDRSAAPGNFSGEEVRLASDHPKKPAGRKKFRETRHPVYRGVRLRDSGKWVCEVREPRKKSRIWLGTFPTADMAARAHDVAALALRGQSACLNFADSAWRLPVPASPNTKDIQKAATKAAEAFQLLESEDVMSGNEKKLHSEEGVLYDEEDIFGMPGLLANMAEGMLLSPPECSGDLYAGLDDGNLDAYASLWSYSM

SEQ ID NO: 5 - DNA, 690 bp, Eucalyptus sp.
atggaccacttcttctcttcttactccgatcccagctcctgcagcctcgactttgctgaagcgtcgtcctcctcgtcgccgctgtccgatggcaggagtgctatggtgcccgggaacttttctgatgaggaggtgctcctggcgtcgcaccagccgaagaagcgcgccgggcggaagaagttccaggagacgcgccaccccgtgtaccgcggggtgcggcggcgaagctcgggcaagtgggtctgcgaggtccgcgagcccaacaagaagtcgaggatctggctgggcaccttccccaccgcggagatggctgcgagggcgcacgacgtggcggcgctcgcgctgaggggccggtccgcctgcctcaacttcgcggactccgcgtggcggctgcccgcgccggcgtcggcggacgcaaaggacatacagcaggcggcggcccaggctgcggaggcgttccggccggcggagtccgaggctgaggacgtgatgtcggggtacgagaagaagtcgccttcggaggagggaatgctgtacgacgacgaggacgtcttcgggatgccggggttactcacgaatatggcggaggggatgctcttgcctccaccacaatgcggcggagatgggtacggcggagaggacgacgggaatctggatgcatacgtgtcgttatggaactattccatgtag

SEQ ID NO: 6 - protein, 229 aa, Eucalyptus sp.
MDHFFSSYSDPSSCSLDFAEASSSSSPLSDGRSAMVPGNFSDEEVLLASHQPKKRAGRKKFQETRHPVYRGVRRRSSGKWVCEVREPNKKSRIWLGTFPTAEMAARAHDVAALALRGRSACLNFADSAWRLPAPASADAKDIQQAAAQAAEAFRPAESEAEDVMSGYEKKSPSEEGMLYDDEDVFGMPGLLTNMAEGMLLPPPQCGGDGYGGEDDGNLDAYVSLWNYSM

SEQ ID NO: 7 - DNA, 989 bp, Eucalyptus sp.
atggcagcccccgggaacttctccgacgaggaggcgcgcctcgcgtcccaccacccaaagaagcgcgccgggaggaagaagttccgggagacgcgccaccccgtgtaccggggagtgcggctgcgtgactcgggcaagtgggtctgcgaggtccgcgagcccagaaagaagtcgaggatctggctcggcaccttccctactgtggagatggcggcgagggcgcacgatgtggcagcgctcgcgctgaggggccggtctgcctgcctcaacttcgcagactccgcgtggcggctgcccgtgccagcgtcggcagacaccaaggacatacagaaggcggcggccaaggccgcggaggcattccagccagtggagtccgagtccgaggacgtgatgtcgggggacgagaagaagtcgccttcggaggagggaatgctgtacgacgacgaggacgtcttcgggatgccggggttgctcatgaatatggcggaggggatgctcttgcctccaccacgatgcggcggagatgggtacggcggagaggacgacgggaatctggatgcatacgtgtcgttatggagctattccatgtagtcatttctcaagttcagttatactttttgtggttagggacgactgggatgccgtactaaaatattttttgatggagtgcggatgaaaggcgaaggtgggtacgaaagcagaaggtattttcggaagccaatttttggggcatgccgttaggtagataatagagactgcacggctcttctgatgttgggcctctgatggaagaataaaagtaggccatgttcacggcagcatcgggcgttgcatatagttgataaagagactgcccgagtcatcatgatcttcgaatgtaaatcatgtaccatgtacaaggcaaagagtgtgctcttgtaattcttatggtttgtttgggttcaaatgctttttgctctggcccattaagacgataaattgaccatccc

SEQ ID NO: 8 - protein, 196 aa, Eucalyptus sp.
MAAPGNFSDEEARLASHHPKKRAGRKKFRETRHPVYRGVRLRDSGKWVCEVREPRKKSRIWLGTFPTVEMAARAHDVAALALRGRSACLNFADSAWRLPVPASADTKDIQKAAAKAAEAFQPVESESEDVMSGDEKKSPSEEGMLYDDEDVFGMPGLLMNMAEGMLLPPPRCGGDGYGGEDDGNLDAYVSLWSYSM

SEQ ID NO: 9 - DNA, 11078 bp, Artificial Sequence
  Description of Artificial Sequence Synthetic polynucleotide
gtttacccgccaatatatcctgtcaaacactgatagtttaaacttttaattaaggtaccgggccccccctcgaggtcgacggtatcgataagcttggttgctatggtagggactatggggttttcggattccggtggaagtgagttgggaggcagtggcggaggtaagggagttcaagattctgggaactgaagatttggggttttgcttttgaatgtttgtgtttttgtatgatgcctctgtttgtgaactttgatgtattttatctttgtgtgaaaaagagattgggttaataaaatatttgcttttttggataagaaactcttttagcggcccattaataaaggttacaaatgcaaaatcacgttagcgtcagatatttaattattcgaagatgattgtgatagatttaaaattatcctagtcaaaaagaaagagtaggttgagccagaaacagtgacatctgttgtttgtaccatacaaattagtttagattattggttaacatgttaaatggctatgcatgtgacatttagaccttatcggaattaatttgtagaattattaattaagatgttgattagttcaaacaaaaattttatattaaaaaatgtaaacgaatattttgtatgttcagtgaaagtaaaacaaattaaattaacaagaaacttatagaagaaaatttttactatttaagagaaagaaaaaaatctatcatttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcgggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagtcacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttggcgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaccatggactcattttctgccttttctgaaatgtttggctccgattacgagtctccggtttcctcaggcggtgattacagtccgaagcttgccacgagctgccccaagaaaccagcgggaaggaagaagtttcgtgagactcgtcacccaatttacagaggagttcgtcaaagaaactccggtaagtgggtgtgtgagttgagagagccaaacaagaaaacgaggatttggctcgggactttccaaaccgctgagatggcagctcgtgctcacgacgtcgccgccatagctctccgtggcagatctgcctgtctcaatttcgctgactcggcttggcggctacgaatcccggaatcaacctgtgccaaggaaatccaaaaggcggcggctgaagccgcgttgaattttcaagatgagatgtgtcatatgacgacggatgctcatggtcttgacatggaggagaccttggtggaggctatttatacgccggaacagagccaagatgcgttttatatggatgaagaggcgatgttggggatgtctagtttgttggataacatggccgaagggatgcttttaccgtcgccgtcggttcaatggaactataattttgatgtcgagggagatgatgacgtgtccttatggagctattaaaattcgatttttatttccatttttggtattatagctttttatacatttgatccttttttagaatggatcttcttctttttttggttgtgagaaacgaatgtaaatggtgcggccgctctagacaggcctcgtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactggtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcaggagcccgggctctcgagtaaaacataattttggcagtaaaaaagtgaattctattgttttgaaaacaaaacaaaatacaggaagcgtgattgtggggttgttgttgaacttgcccgggcaaaagaagaatgattagcggtagaggagttagtagttacgttcaactaaatgcgtgactaaattatttatcctccgccatggaagcaggtgattcacacacaacttgctgcacacattgctctcaaacctttcctataaatatccgtagcaggggctgcgatgatacacaacgcatttaatcaaactactttgattactttctgtgggttctactttctttgaatagtcagttctgctgtttttagaagatttatggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggacgagtatcagacctttacaaaaatcagataacgaaaaaaacggcttccctgcgggaggccgtttttttcagctttacataaagtgtgtaataaatttttcttcaaactctgatcggtcaagagctcttctgagagacaatacatacatgtctctgatgttgtaactttactaccaaaacctataaagattggcttatttcgttctattggatatgtatcatcattactggtaaatcaagtttctttctaataatgtagaagatcagaaaatccataagaagatatcaacatttgagttctatggtaaattgaattatatcaacttagttgcaatgattcattcttgactgatgcattgatggcttatcaaaccagtttcaaaattcgattagatagggcccatttaaatgcggccgcttggcgcgccgtcaacggatcaggatatccttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcacgtaggggggatccactagttctagagcggccgtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagttgtctatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattgtggtttgtgtttccatattgttcctctcccattgatcgtattaagaaagtatgatggtgatgtcgcagccttccgctttcgcttcacggaaaacctgaagcacactctcggcgccattttcagtcagctgcttgctttgttcaaactgcctccattccaaaacgagcgggtactccacccatccggtcagacaatcccataaagcgtccaggttttcaccgtagtattccggaagggcaagctcctttttcaatgtctggtggaggtcgctgatacttctgatttgttccccgttaatgactgcttttttcatgtgcggctcctttcttatgattttattctatcagtttaccatttttctcttcagaaatggccggattctgccccggtttcagctggacgatgtcctcctgtctcggacggctgcaaattggaccacaattatggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggatctggggaaccctgtggttggcatgcacatacaaatggacgaacggataaaccttttcacgcccttttaaatatccgattattctaataaacgctcttttctcttag